(19) United States
(12) Patent Application Publication
(10) Pub. No.: US 2005/0228322 A1
(43) Pub. Date: Oct. 13, 2005

(54) Machine and process for lining and/or cushioning orthopedic casts and other orthopedic devices

(76) Inventor: John Louis Stanton, Clarksville, TN (US)

Correspondence Address: DAVID DOUGLAS WINTERS, 2277-C SUITE 237, WILMA RUDOLPH BLVD, CLARKSVILLE, TN, 37040-5898, US

(21) Appl. No.: 10/819,650

(22) Filed: Apr. 7, 2004

(51) Int. Cl. A61L 15/00

(52) U.S. Cl. 602/3; 602/75

(57) ABSTRACT

A device comprised of hydrophobic synthetic polymers used for cushioning and/or protecting a part from chaffing, pressure point injury, damage or discomfort from an orthopedic or non-orthopedic, cast, compression dressing, and/or brace, and to protect against a cast saw blade.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### LEXICON

Use of the word “impervious” in this patent is intended to mean “not affected by.”

### BACKGROUND OF THE INVENTION

This invention relates generally to the field of orthotic, or orthopedic device liners and more specifically to a machine and process for lining and/or cushioning orthopedic casts and other orthopedic devices.

The method presented in this patent has been exercised on volunteer human subjects with unqualified success. Properly administered, it provides for significantly improved comfort, freedom of activities and freedom from inflamation or infection over previously known technologies.

The following patents and product literature are representative of the previously known art in the field.

U.S. Pat. No. 4,194,041, describes a waterproof and breathable polytetrafluoroethylene (hereinafter PTFE) laminate which allows high water vapor transmission even under adverse climatic conditions. Such laminates in modified forms are commercially available from W. L. Gore & Associates. The waterproof and breathable laminate described in this patent consists of a flexible outer layer of micro porous hydrophobic material, such as porous, expanded PTFE, (hereinafter ePTFE) and a second interior layer of continuous hydrophilic elastomeric material attached to the interface of the outer layer. The interior layer does not permit passage of liquid water. The material is specified for use in the fabrication of waterproof, breathable garments, tents, and the like.

U.S. Pat. No. 4,989,593 discloses a rigid orthopedic cast or splint which includes padding treated with a fluoro-chemical or silicone. The padding is able to shed water rapidly promoting comfort for the user.

U.S. Pat. No. 5,016,622, discloses an orthopedic cast and padding assembly having a water impermeable, water vapor permeable membrane in contact with the skin, a resilient padding layer of conventional material, and a plaster or resin/glass fiber outer immobilizing layer. The assembly is said to increase the comfort of a wearer and reduce bacteria contained within the cast.

Another cast material is found in U.S. Pat. No. 5,102,711, This patent discloses a flexible, breathable, composite having a middle layer of conventional man-made or natural fiber padding and a top and bottom layer comprising a sheet of water impermeable, water vapor permeable film bonded to the middle layer. Preferably the top and bottom layers comprising (a) a flexible first sheet of hydrophobic material; and (b) a continuous hydrophilic sheet attached to or penetrating the first sheet forming a barrier to passage of fluids. The composite material is useful in applications such as liners under orthopedic casts and as thermal insulation in apparel.

U.S. Pat. No. 5,397,628, discloses a body protection material having an inner layer of ePTFE laminated to an outer layer of substantially air impermeable cellular rubber.

There exists a need, unfilled by the above patents, and primarily with respect to orthotic or orthopedic purposes, for a means of providing, and an orthopedic system using, effective, inexpensive, fast drying, non-allergenic, waterproof protection and padding, that can be used in direct contact with the skin of a wearer. Applications include various garments such as sleeves, socks and gloves, liners for orthotic devices such as casts and braces and orthotic devices using such liner/padding material.

Cast and brace liner applications are exemplary of the need for waterproof fabrics and of the lack of suitable fabrics in the prior art to fulfill these needs. Various orthopedic and orthotic devices and processes are commonly used to reinforce or immobilize a portion of the body of the wearer, usually a limb or joint. Casts are commonly employed when treating a bone fracture.

In applying a conventional cast; it is common practice to first wrap or otherwise cover the body member being treated with a protective fabric made of a material such as cotton or polyester knit. The thus protected body member is then covered with padding, also of cotton or polyester, to provide a soft, resilient cushion/lining.

The padding is then over-wrapped with a cast of plaster and woven cloth, or of fiberglass material in the form of heat-softened or curable organic polymer resins, coated on glass fibers formed into fabrics, which are wrapped about the body member and thereafter hardened by cooling or by application of specific energy such as ultra-violet radiation.

Similarly, art is also known for applying various braces and orthotic devices to the body of a wearer, mainly to the limbs and joints. In some applications, these braces are compression members. Others may incorporate some type of range of motion limiting device. Such devices are widely used in therapeutic applications and are also widely used in connection with athletic activities, particularly to protect knee joins from injury.

Use of orthopedic casts, orthotic devices and braces often causes discomfort to the wearer. Conventional neoprene braces often cause an adverse allergic reaction resulting in skin irritation. These braces are also known to be excessively abrasive where they contact the skin and are likewise known to migrate from their desired positions during activity. The combination of their abrasive character and the migration frequently results in skin irritation and laceration. Perspiration coupled with a lack of circulation further allows irritation and maceration of the skin under the cast, device, or brace, which may result in the growth of bacteria and skin erosion. If untreated, this condition can lead to infection.

Additionally, while the fiberglass cast shell may be impervious to water, the cotton and cloth liner is not. It holds water, dries slowly, and retains moisture against the skin. It is also weakened and tends to clump and bunch up when wetted. The accompanying lack of air to dry the skin could also encourage maceration under the cast and opening it to attack by bacteria.

Furthermore, unpleasant odor may develop due to retention of body oils and perspiration inside the cast. Ability to immerse or irrigate the interior of the cast without damage can be valuable in helping to prevent this.

Skin erosion and subsequently formed pustules that emanate from blocked hair follicles can cause extreme discomfort under a conventional cast. Additionally, urine soaked cast linings, particularly in the case of casts worn by infants, have wet padding in constant contact with the skin, resulting in ulcerations.

Previously taught technologies do not overcome the problem of retained dampness once liquid gets inside the lining and reaches the user's skin. None of the protective liner systems do, nor should, provide a water tight seal against immersion. Additionally, in conditions of immersion, the previously taught and otherwise valuable quality of being water impermeable while water vapor permeable becomes moot because liquid water will inevitably enter the interior of the liner by bypassing the edges of the protective materials. The problem to be addressed then becomes one of quickly getting the skin dry again.

Without the ability to efficiently dry the interior of orthopedic or orthotic devices, particularly casts, immersing, cleansing or rinsing the interiors becomes unfeasible.

### BRIEF SUMMARY OF THE INVENTION

A primary object of the invention is that it may be immersed in water without ill effect.

Another object of the invention is that it will quickly shed water.

Another object of the invention is that it be non-absorbent, resisting water vapor, liquid water, and odors.

Yet another object of the invention is to promote quick drying of dampness or perspiration in the interior of an orthopedic cast or other orthopedic device.

Another object of the invention is to provide superior, resilient protection from chaffing and/or pressure points in casts and other orthopedic devices.

A further object of the invention is to allow circulation of air inside and throughout the orthopedic device.

Still, yet another object of the invention is that it have superior strength and resilience over cotton liners that are presently the dominant lining and cushioning material of choice.

Another object of the invention is that it stretch and conform to the topography of the surface it covers.

Another object of the invention is that it resist migration and retain position as applied.

Another object of the invention is that it protect against contact with any cast-saw used in removing orthopedic casts.

A further object of the invention is to allow re-use as wrapping under compression dressings in the event of re-wrapping or change of said dressings.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a machine comprised of hydrophobic synthetic polymers configured in strip(s), swath(s), tube(s), sleeve(s), glove(s), or other forms compatible with use for cushioning and/or protecting a human, animal, or plant subject from chaffing, pressure point injury, damage or discomfort from a protective device, orthopedic cast, compression dressing, and/or other orthopedic or non-orthopedic device, and to protect against a cast saw blade in the removal process, and;

In accordance with a preferred embodiment of the invention, there is disclosed a process wherein said machine is employed to cushion and/or protect a human, animal, or plant subject from chaffing and pressure point injury, damage or discomfort from a protective device, orthopedic cast, compression dressing, and/or other orthopedic or non-orthopedic device and against a cast saw blade in the removal process.

### BRIEF DESCRIPTION OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1A is a drawing of the device fabric (10) used in the preferred mode with some of the scrim (20) exposed. It is cut on the bias (30) into strip form, also in accordance with the preferred mode.

FIG. 2A is a drawing of the device (100) in preferred mode being applied to a hand and forearm.

FIG. 3A is a drawing of the device (100) in preferred mode applied to a hand and forearm and having a fiberglass cast shell (200) in the process of being wrapped over it.

FIG. 4A is a drawing of the device (100) in preferred mode in initial stage of being applied to a foot and lower leg.

FIG. 5A is a drawing of the device (100) in preferred mode as applied to a foot and lower leg and ready to receive a cast shell.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

In the preferred embodiment, the present device uses materials comprised of hydrophobic synthetic polymers, in example, PTFE or Polyvinylchloride (PVC) in closed cell foam mode, for cushioning and/or protecting a subject from chaffing, pressure point injury, damage or discomfort from an orthopedic or non-orthopedic protective device such as a cast, compression dressing, or brace, and to protect against a cast saw blade in the removal process.

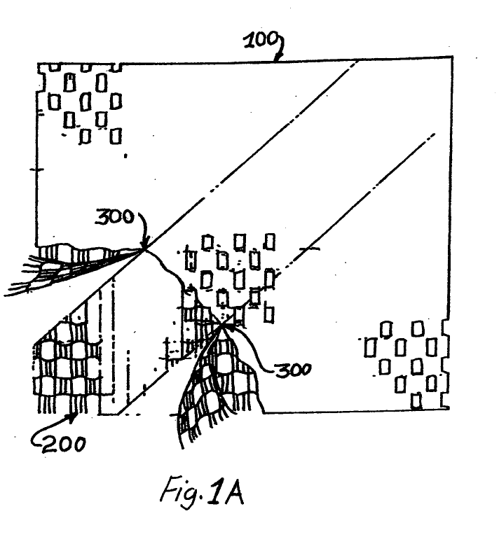

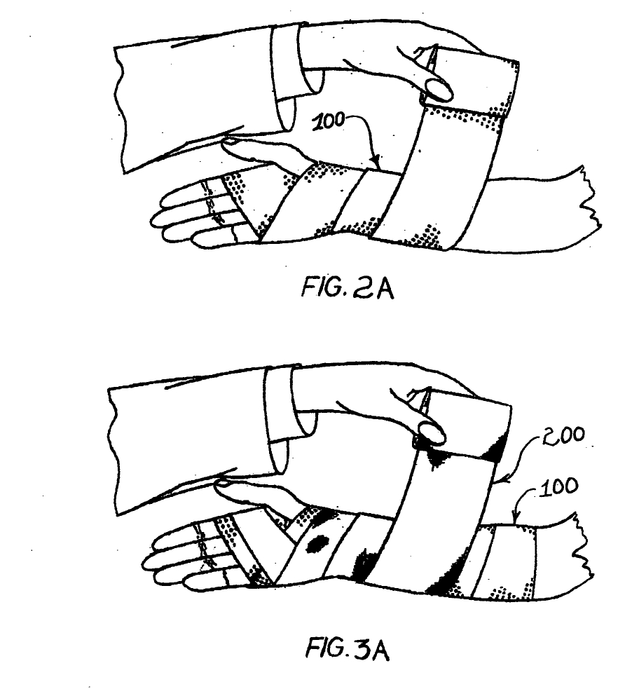

In the preferred embodiment, the hydrophobic synthetic polymers are composed about a grid core or scrim, (See FIG. 1A, item 20) giving resilience, flexibility, and elasticity. The material is configured with numerous perforations, holes, or openings through it, and prepared before application by cutting it, on the bias, into strips, which is to say cutting along a line not parallel to the true or apparent warp, woof, texture, or grid lines of the core grid or scrim. This gives it significantly improved flexibility and elasticity to conform to the topography of the part of the body it protects and improves it's resistance to migration while in use.

Additional padding is then wound up the forearm in a spiral fashion with about 20% to 30% overlap, and then cut and taped to itself near the elbow.

Referring to FIG. 3A, a cast (200) is then applied by wrapping fiberglass around the hand and forearm in a fashion conforming to previously standardized methods. Redundant interior padding is then folded back at each end and held under the fiberglass wrap.

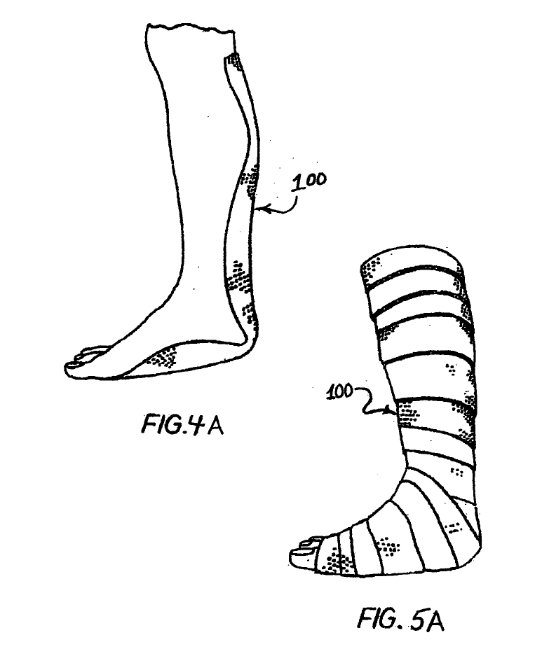

Referring to FIG. 4A, for application to a foot and ankle, a length of the material composed and prepared as above is run up the back of the calf from toes to knee. Additional wrap is placed around the foot and calf from the toes to knee with 20% to 30% overlap. In FIG. 5A, the device is ready for fiberglass casting material to be wrapped over the padding and redundant padding is folded back and included in the cast at each end.

When used as padding under compression dressings, the padding material needs not be discarded upon adjustment or re-application of the dressing, but may be adjusted and use continued in place.

In alternative form, the bias cut material may be joined at the edges in approximately straight lines to form a tube or sleeve. In more detailed configuration it may be formed into a glove, sock, cap or other piece of apparel.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1-11. (canceled)

12. A machine comprising cushion material composed, essentially, of hydrophobic synthetic polymers or of breathable polytetraflouroethylene, configured in strip(s), swath(s), tube(s), sleeve(s), glove(s), or other forms as means of padding, cushioning and or, or, protecting body part from discomfort, injury or damage from a protective device, orthopedic cast, brace and or, or, compression dressing.

13. A machine as in claim 12, wherein the cushion material is composed about a grid core or scrim.

14. A machine as in claim 12, wherein the cushion material is configured with numerous perforations, holes, or openings.

15. A machine as in claim 12, wherein the cushion material is prepared and/or shaped by cutting and/or joining it on the bias, which is to say along a line not parallel to the true or apparent warp, woof, texture, or grid lines of the core grid or scrim.

16. A process wherein the machine as in claim 12 is employed s a means to pad, cushion and/or protect a human, animal, or plant subject from a cast saw blade in an orthopedic device removal.

17. A process wherein the machine of claim 12 is employed as a means for lining a protective device, orthopedic cast, compression dressing, and/or other orthopedic or non-orthopedic device.

18. A process wherein the machine of claim 12 is employed as a means to promote quick-drying of dampness and/or perspiration in the interior of a protective device, orthopedic cast, compression dressing or other orthopedic or non-orthopedic device.

19. A process wherein the machine of claim 12 is employed as a means to permit unprotected liquid immersion of a body part while in a protective device, such as an orthopedic cast, compression dressing, and/or other orthopedic or non-orthopedic device.

20. A process wherein the machine of claim 12 is employed as a means to permit improved ventilation and/or air flow within and through the interior of the protective device, orthopedic cast, compression dressing, and/or other orthopedic or non-orthopedic device.

21. A process wherein the machine of claim 12 is employed as a means for it's own reuse after adjustment and/or changing of the protective device, orthopedic cast, compression dressing, and/or other orthopedic or non-orthopedic device.

22. A machine comprising an immobilizing orthopedic cast, comprising:
(a) a machine comprising protective/cushion material composed essentially of hydrophobic synthetic polymers or essentially of breathable polytetraflouroethylene, as a means of padding, and/or protecting a body part against chaffing, pressure point injury, damage or discomfort from an orthopedic cast, and to protect against a cast saw blade in a cast removal process; and
(b) an outer immobilizing layer of water impervious material positioned around said machine.

23. A method of protecting, or cushioning a body part from discomfort, injury or damage from a protective device, orthopedic cast, brace and or, or, compression dressing, comprising;
(a) Providing padding material composed essentially of hydrophobic synthetic polymers or essentially of breathable polytetraflouroethylene, configured in strip(s), swath(s), tube(s), sleeve(s), glove(s)
(b) Placing the body part to be buttressed or protected in a position appropriate to maintain good support or fracture reduction as needed.
(c) wrapping or covering the part with a length of said padding material
(d) applying a cast or orthopedic device to the essentially padding covered part.

24. A method as in claim 23 also comprising provision of the cushion material in a form composed about a grid core or scrim;

25. A method as in claim 23 also comprising configuring the cushion material with numerous perforations, holes, or openings;

26. A method as in claim 23 also comprised of preparing or shaping the cushion material by cutting and, or or, joining it on the bias, which is to say along a line not parallel to the true or apparent warp, woof, texture, or grid lines of the core grid or scrim;
